SEQUENCE LISTING (33 sequences)

SEQ ID NO: 1 - protein, 361 aa, synthetic construct
MTDLTPFPPLEHLEPDEFADLVRKAIKRDPQAGAHPAIQSAISHFQDEFVRRQGEWQPATLQRLRNAWNVFVRWCTHQGIPALPARHQDVERYLIERRNELHRNTLKVHLWAIGKTHVISGLPNPCAHRYVKAQMAQITHQKVRERERIEQAPAFRESDLDRLTELWSATRSVTQQRDLMIVSLAYETLLRKNNLEQMKVGDIEFCQDGSALITIPFSKTNHSGRDDVRWISPQVANQVHAYLQLPNIDADPQCFLLQRVKRSGKALNPESHNTLNGHHPVSEKLISRVFERAWRALNHETGPRYTGHSARVGAAQDLLQEGYSTLQVMQAGGWSSEKMVLRYGRHLHAHTSAMAQKRRQR

SEQ ID NO: 2 - protein, 9 aa, synthetic construct
YPYDVPDYA

SEQ ID NO: 3 - protein, 6 aa, synthetic construct
KKKRKV

SEQ ID NO: 4 - DNA, 34 bp, synthetic construct
  source 1..34: mol_type = other DNA
aataggtctgagaacgcccattctcagacgtatt

SEQ ID NO: 5 - DNA, 20 bp, synthetic construct
  source 1..20: mol_type = other DNA
gtgtgatgttgcaagtgtgg

SEQ ID NO: 6 - DNA, 20 bp, synthetic construct
  source 1..20: mol_type = other DNA
aatgcttccatcaaacgagt

SEQ ID NO: 7 - DNA, 17 bp, synthetic construct
  source 1..17: mol_type = other DNA
ccccgctaccccaatcc

SEQ ID NO: 8 - DNA, 24 bp, synthetic construct
  source 1..24: mol_type = other DNA
ttagcttttttgggtgatttttcc

SEQ ID NO: 9 - DNA, 18 bp, synthetic construct
  source 1..18: mol_type = other DNA
tgggcgtggtgcctaaaa

SEQ ID NO: 10 - DNA, 19 bp, synthetic construct
  source 1..19: mol_type = other DNA
tgtctttcagtagcaagct

SEQ ID NO: 11 - DNA, 16 bp, synthetic construct
  source 1..16: mol_type = other DNA
gcctgcccctggcaat

SEQ ID NO: 12 - DNA, 20 bp, synthetic construct
  source 1..20: mol_type = other DNA
ggaagtgtgatgttgcaagt

SEQ ID NO: 13 - DNA, 17 bp, synthetic construct
  source 1..17: mol_type = other DNA
caatgctggagcccatc

SEQ ID NO: 14 - DNA, 1167 bp, synthetic construct
  source 1..1167: mol_type = other DNA
atgctagccaccatgtatccctacgacgtgcccgactacgccaagaaaaagcggaaagtgaccgatctgaccccatttcctcctctggaacacctggaacctgacgagttcgccgatctcgtgcggaaggccatcaagagagatcctcaggctggcgcccatcctgccatccaatctgccatcagccacttccaggatgagttcgtgcggagacaaggcgaatggcagcctgccacactgcagagactgagaaacgcctggaatgtgtttgtgcggtggtgcacccaccagggcattccagcattgcctgccagacaccaggacgtggaaagatacctgatcgagcggcggaacgagctgcacagaaacaccctgaaagtgcacctgtgggccatcggcaagacccacgttatcagcggcctgcctaatccatgcgctcacagatacgtgaaagcccagatggcccagatcacacaccagaaagtgcgcgagagagagcggattgaacaggcccctgccttcagagagtccgacctggatagactgaccgagctttggagcgccaccagaagcgttacccagcagagggacctgatgatcgtgtccctggcctacgagacactgctgcggaagaacaatctggaacagatgaaagtgggcgacatcgagttctgccaggatggctctgccctgatcacaatccccttcagcaagaccaaccacagcggcagagatgacgtgcggtggatctctcctcaggtggccaatcaggtgcacgcctatctgcagctgcccaacatcgatgccgatcctcagtgcttcctgctgcagagagtgaagagaagcggcaaggccctgaatcctgagagccacaataccctgaacggccaccatcctgtgtccgagaagctgatctcccgggtgttcgagagagcttggagagccctgaaccacgagacaggccctagatacacaggccacagcgctagagttggcgccgctcaagatctgctgcaagagggctacagcaccctgcaagttatgcaagccggcggatggtccagcgagaagatggtgctgagatacggcagacatctgcacgcccacacaagcgccatggctcagaagagaaggcagcggagcagagtggaagagttcagcctctga

SEQ ID NO: 15-19 - skipped (5 entries)

SEQ ID NO: 20 - protein, 360 aa, synthetic construct
MTDLTPFPPLEHLEPDEFADLVRKAIKRDPQAGAHPAIQSAISHFQDEFVRRQGEWQPATLQRLRNAWNVFVRWCTHQGIPALPARHQDVERYLIERRNELHRNTLKVHLWAIGKTHVISGLPNPCAHRYVKAQMAQITHQKVRERERIEQAPAFRESDLDRLTELWSATRSVTQQRDLMIVSLAYETLLRKNNLEQMKVGDIEFCQDGSALITIPFSKTNHSGRDDVRWISPQVANQVHAYLQLPNIDADPQCFLLQRVKRSGKALNPESHNTLNGHHPVSEKLISRVFERAWRALNHETGPRYTGHSARVGAAQDLLQEGYSTLQVMQAGGWSSEKMVLRYGRHLHAHTSAMAQKRRR

SEQ ID NO: 21 - protein, 415 aa, synthetic construct
MAKTTVKLTPTKIKSSKPKDKEYNLFDGDGLRLRIKPNGSKHWILNYYRPSNRKRANLSLGKYPDLSLANARKMTLEAKELLAQGIDPQEERKRHQLEQKANHEHTFINVTTKWFEIKKDDVTPDYAVDIWRSLELHIFPQLSDVPVRDITAPQVIELLKPIEAKGSLETVKRLAQRLNEVMNYATNCGLVQANPLTGIKAAFKKPKKENMAALTPAELPELMGAIANASIKRTTRCLIEWQLHTMTRPSEASGARWEEIDWDEKVWTIPPERMKKRREHRIPLTEQMLELLEVIKPISGHREFIFPSDRDPKKPCNSQTANMALKRMGFAGRLVSHGLRSLASTTLNEQGFEPDLIEAALAHADDNQVRSAYNRTDYLERRRPMMCWWSGHIEEAAKGSLSVTGMKQLKVINVD

SEQ ID NO: 22 - protein, 361 aa, synthetic construct; identical to SEQ ID NO: 1

SEQ ID NO: 23 - protein, 320 aa, synthetic construct
MPEKSPFLREVCEHMRLSGYSIRTEKSYLYWIRSYIFYHEKRHPMQMGSEEVIQFLSYLANSRNVAINTQKVALNALVYLYQAFLKIELGELGFRHATKQRQLPVVITPNEVKQVLSHLNGKEKLIVELLYGSGLRISECLRLRVQDIDLSNLSITVRDGKGKKDRQTLLSKQCVDVLKLLIEQAEQVQIADNKHGIGPSLPYAIHKKYPNAFRKLAWMFIFPSPSISHSQYNSDHPSRHHLHSSVIRKALSRAVAKTTINKKVTCHTFRHSFATHLLQAGRDIRSVQELLGHNDVKTTQIYTHVIGQHYAGTTSPLDNL

SEQ ID NO: 24 - protein, 321 aa, synthetic construct
MPEKSPFLREVCEHMRLSGYSIRTEKSYLYWIRSYIFYHEKRHPMQMGSEEVIQFLSYLANSRNVAINTQKVALNALVYLYQTFLKIELGELGFRHATKQRQLPVVITPNEVKQVLSHLNGKEKLIVELLYGSGLRISECLRLRVQDIDLSNLSITVRDGKGKKDRQTLLSKQCVDVLKLLIEQAEQVQIADNKHGIGPSLPYAIHKKYPNAFRKLAWMFIFPSPSISHSQYNNDHPSRHHLHSSVIRKALSRAVAKTTINKKVTCHTFRHSFATHLLQAGRDIRSVQELLGHNDVKTTQIYTHVIGQHYAGTTSPLDNLN

SEQ ID NO: 25 - protein, 309 aa, synthetic construct
MSESAIPLPNGLQSPLNRFYEYLRSEKGLSLHTQRNYKQQLETMAQHLVTMGLKDWTQVDAAWVRQLASKGMRDGMKASSLATRLSALRSFFDFLILRGELTANPAKGVSAPRKKRPLPKNLDVDEVGQLLEVNEDDPLAIRDRAMMELMYGAGLRLAEMVSVDVRDVSLSSGEIRVVGKGDKERKVPFAGMAEEWVAKWLKVRGVLANTDEPALFVSKLGVRISHRNVQKRMAEWGQKQSVASHISPHKLRHSFATHMLESSNNLRAVQELLGHENISTTQIYTHLDFQHLADVYDQAHPRAKKKESD

SEQ ID NO: 26-29 - skipped (4 entries)

SEQ ID NO: 30 - DNA, 34 bp, synthetic construct
  source 1..34: mol_type = other DNA
ataacttcgtatagcatacattatacgaagttat

SEQ ID NO: 31 - DNA, 34 bp, synthetic construct; identical to SEQ ID NO: 4
  source 1..34: mol_type = other DNA

SEQ ID NO: 32 - DNA, 20 bp, synthetic construct
  source 1..20: mol_type = other DNA
agcctctctcatctcacagt

SEQ ID NO: 33 - DNA, 19 bp, synthetic construct
  source 1..19: mol_type = other DNA
tgtctttcagtagcaagct

(19) United States
(12) Patent Application Publication
(10) Pub. No.: US 2023/0265382 A1
(43) Pub. Date: Aug. 24, 2023

(54) PRODUCTION SYSTEM FOR HELPER-DEPENDENT ADENOVIRUS

(71) Applicant: University of Iowa Research Foundation, Iowa City, IA (US)

(72) Inventors: Samuel M. Young, JR., Iowa City, IA (US); Stacia Phillips, Iowa City, IA (US)

(21) Appl. No.: 17/984,575

(22) Filed: Nov. 10, 2022

Related U.S. Application Data
(60) Provisional application No. 63/263,857, filed on Nov. 10, 2021.

(51) Int. Cl. C12N 5/071; C07K 14/075; C12N 15/86

(52) U.S. Cl. CPC C12N 5/0602; C07K 14/075; C12N 15/86; C12N 2710/10041

(57) ABSTRACT

Methods to produce helper dependent adenovirus, and a cell, vector and kit useful in that regard, are provided.

### CROSS-REFERENCE TO RELATED APPLICATIONS

This application claims the benefit of the filing date of U.S. application No. 63/263,857, filed on Nov. 10, 2021, the disclosure of which is incorporated by reference herein.

### STATEMENT OF GOVERNMENT RIGHTS

This invention was made with government support under DC014093, NS 110742, and DC018242 awarded by the National Institutes of Health. The government has certain rights in the invention.

### INCORPORATION BY REFERENCE OF SEQUENCE LISTING

A Sequence Listing is provided herewith as an xml file, “2334837.xml” created on May 16, 2023, and having a size of 27,439 bytes. The content of the xml file is incorporated by reference herein in its entirety.

### BACKGROUND

Recombinant viral vectors are key research tools for elucidating the cell-type specific mechanisms that control cellular activity in vivo (Nectow & Nestler, 2020; Chan et al., 2018). Although Adeno-associated virus (AAV) vectors (rAAV) and Lentiviral viral vectors (LVV) are the most widely used viral vectors in both the basic research and clinical setting, they have a relatively small packaging capacity of about 5 kb and about 9 kb, respectively (Li & Samulski, 2020; Anguela & High, 2019). Their small packaging capacity results in limitations in addressing research questions centered on proteins with large coding sequences or that require the delivery of transgene cassettes. Although not as widely-used as rAAV and LVV, Helper-dependent adenovirus (HdAd) vectors have had success in basic research and clinical applications (Brunetti-Pierri & Ng, 2017). HdAd vectors lack all viral genes, have a packaging capacity of about 36 kb (Palmer & Ng, 2011a) and are non toxic and provide long-term transgene expression (Kim et al., 2001; Toietta et al., 2005; Schmitt et al., 2014; Rastall et al., 2016). More importantly, they enable the ability to address long-standing research questions not possible with the limited packaging capacity of rAAV or LVV (Kim et al., 2001; Rastall et al., 2016; Kiang et al., 2006).

Genetic strategies relying on cell type-specific Cre or Flp recombination to induce expression from rAAV or LVV vectors are important tools in elucidating the cellular and molecular mechanisms controlling cellular activity in vivo and organismal behavior (Weissman & Pan, 2015; Atasoy et al., 2008; Fenno et al., 2017). However, the limited packaging capacity of these vectors results in the inability to probe many molecules and their signaling cascades in their native context.

### SUMMARY

The dependency on Cre or Flp recombination prevents the production of helper dependent adenovirus (HdAd) vectors containing, for example, loxP or FRT sites. Therefore, there is a need for a system to produce HdAd in a Cre or Flp-independent manner. In one embodiment, as described herein, a Vika recombinase (Vika hereinafter), e.g., from Vibrio coralliilvticus, that recognizes a 34 bp palindromic target site (vox) and exhibits efficient site-specific recombination in both bacterial and mammalian cells (Karimova et al., 2013), may be employed to produce HdAd. Vika has little or no activity on pseudo vox sites that are present in the mammalian genome and has significantly lower genotoxicity and cytotoxicity than Cre (Karimova et al., 2013).

A Vika-based HdAd production system overcomes the limitations of using HdAd in research applications, and so a HdAd production method was designed using Vika/vox recombination. To do so, in one embodiment, a producer cell line that expresses Vika, and a helper virus (HV) containing the Ad ITRs with a flanked by vox sites, were employed. This system, called VikAD, supports efficient replication of HdAd vectors to high titers with low levels of contaminating HV, e.g., less than 0.4% HV prior to density purification or less than 0.01% HV density purification.

For example, VoxHV, Vika293 cells and an HdAd FLEX vector that relies on Cre-dependent excision for transgene expression were prepared and used to demonstrate that HdAd FLEX is compatible with Cre expressing cell lines, a mouse Cre driver line, and with co-injection of an HdAd Cre plus HdAd FLEX virus in the mouse auditory brainstem. In particular, VikAD was used to produce an HdAd FLEX EGFP vector and demonstrate Cre-dependent expression of EGFP in 1) a cell line, 2) a mouse Cre driver line, and 3) using dual delivery of both HdAd Cre and HdAd FLEX EGFP in vivo. The results show that VikAD enables the production of HdAd vectors containing non-vox recombination sites, e.g., loxP sites.

Since VikAd enables the production of HdAd vectors that utilize Cre, Flp, a Cre derivative or other site-specific recombinase excision for transgene expression in vitro and in vivo, VikAD has the ability to expand the utility of HdAd, e.g., for applications that rely on genetic intersectional strategies using Cre and Flp or other site-specific recombinases in vitro and in vivo that are not possible with rAAV or LVV.

Thus, the disclosure provides for a site-specific recombinase-based production system for efficient and scalable production of helper-dependent adenovirus.

In one embodiment, a cell line expressing Vika recombinase, e.g., a mammalian cell line that stably expresses high levels of Vika recombinase, e.g., levels that enable complete excision of the voxed packaging signal, for instance, as determine by PCR amplification, is transfected with a linearized plasmid containing an HdAd genome. Cells are subsequently infected with a helper adenovirus that contains a packaging signal flanked by vox sites and encodes all adenoviral proteins, e.g., those needed for replication and packaging of viral genomes, or encodes all but 1, 2, 3, 4 or 5 or more of the adenoviral proteins. During viral genome replication, the packaging signal in the helper plasmid is excised through the site-specific recognition of the vox sites by Vika recombinase, rendering the helper genome unable to be incorporated into progeny viral particles. The resulting virus produced from the cells contains only HdAd genomes. Thus, the VikAd system includes a helper adenovirus that has been genetically modified to have vox sites flanking the packaging signal and a producer cell line that stably expresses high levels of catalytically active Vika enzyme, which together are useful for the production of HdAds, e.g., those containing loxP, loxP derivative sites, FRT sites, noxP, pox sites or site-specific recombinase recognition sites other than vox.

Thus, the disclosure provides an isolated mammalian cell that stably expresses a recombinant DNA comprising an open reading frame encoding a recombinase having at least 80% amino acid sequence identity to SEQ ID NO:1 and optionally expresses at least one adenovirus protein. In one embodiment, the recombinase has at least 90% amino acid sequence identity to any one of SEQ ID Nos. 20-25. In one embodiment, the isolated cell is a primate cell. In one embodiment, the isolated cell is a human cell. In one embodiment, the open reading frame further encodes a nuclear localization signal. In one embodiment, the isolated cell stably expresses at least one adenovirus protein. In one embodiment, the adenovirus protein that is expressed in the cell includes adenovirus E1A, E1B or both. In one embodiment, the recombinant DNA is expressed from a constitutive promoter, e.g., a heterologous promoter such as a CMV promoter.

Further provided is a recombinant helper adenovirus vector comprising an adenovirus genome comprising two inverted terminal repeats (ITRs) and a packaging signal flanked by a site-specific recombinase recognition site having at least 80% nucleic acid sequence identity to SEQ ID NO:4. In one embodiment, the recombinase recognition site has at least 90% nucleic acid sequence identity to SEQ ID NO:4. In one embodiment, the recombinase recognition site has at least 95°/o nucleic acid sequence identity to SEQ ID NO:4. In one embodiment, the recombinase recognition site is cleaved by a recombinase having at least 80% amino acid sequence identity to SEQ ID NO:1. In one embodiment, the vector is a plasmid.

Also provided is a method to produce helper dependent adenovirus. The method includes contacting an isolated mammalian cell that stably expresses a recombinase having at least 80% amino acid sequence identity to SEQ ID NO:1, a recombinant helper adenovirus vector comprising two ITRs and a packaging signal flanked by a site-specific recombinase recognition site having at least 80% nucleic acid sequence identity to SEQ ID NO:4, and a helper dependent adenovirus vector comprising a genome having two ITRs, a packaging signal, an open reading frame for a gene product of interest and a transcriptional control sequence, wherein if the open reading frame is in reverse orientation relative to the transcriptional control sequence, the open reading frame is flanked by a pair of site-specific recombinase recognition sites other than sites having at least 80% nucleic acid sequence identity to SEQ ID NO:4 or other than sites recognized by a polypeptide having at least 80% amino acid sequence identity to SEQ ID NO:1 or if the transcriptional control sequence is in reverse orientation relative to the open reading frame, the transcriptional control sequence is flanked by a pair of site-specific recombinase recognition sites other than sites having at least 80% nucleic acid sequence identity to SEQ ID NO:4 or other than sites recognized by a polypeptide having at least 80% amino acid sequence identity to SEQ ID NO:1; and collecting helper dependent adenovirus. In one embodiment, the cell is contacted with the recombinant helper adenovirus vector and then with the helper dependent adenovirus vector. In one embodiment, the site-specific recombinase recognition sites other than sites having at least 80% nucleic acid sequence identity to SEQ ID NO:4 are loxP, rox, vloxP, FRT, pox or nox sites. In one embodiment, the transcriptional control sequence is a cell-specific transcriptional control sequence. In one embodiment, the open reading frame encodes a protein. In one embodiment, the open reading frame encodes a therapeutic gene product. In one embodiment, the open reading frame comprises a marker.

In one embodiment, a method to produce helper dependent adenovirus is provided comprising contacting an isolated mammalian cell that stably expresses a recombinase having at least 80% amino acid sequence identity to SEQ ID NO:1, a recombinant helper adenovirus vector comprising two ITRs and a packaging signal flanked by a site-specific recombinase recognition site having at least 80% nucleic acid sequence identity to SEQ ID NO:4, and a helper dependent adenovirus vector comprising a genome having two ITRs, a packaging signal, an open reading frame for a gene product of interest and a transcriptional control sequence; and collecting helper dependent adenovirus.

### BRIEF DESCRIPTION OF FIGURES

FIGS. 1A-1C. Characterization of Vika expression and activity in HdAd producer cells. A) Schematic of the reporter cassette used to indicate the presence of Vika activity. B) Comparison of Vika activity before and after clonal sorting and expansion. 293 pooled Vika cells, and Vika Clone 24 were transfected with reporter plasmid and images were captured 24 hours post-transfection. C) Western blot analysis of Vika expression. Vika protein was detected in total cell lysates using an antibody against the HA epitope tag on the N-terminus of Vika. Actin is the loading control.

FIGS. 2A-2D. VikAd enables excision of VoxHV packaging signal and amplification of HdAd. A) Agarose gel electrophoresis of pVoxHV plasmid (lane 1) and VoxHV genome from purified viral particles (lane 2) after MfeI digestion. Diagnostic bands showing removal of plasmid sequences are shown (arrows). B) Total DNA was purified from infected cell lysates at each passage during HdAd amplification using Vika293 and VoxHV. PCR amplification was performed using primers that flank the HV. 116 cells expressing Cre recombinase infected with a loxP HV serve as a positive control for excision, resulting in a 345 bp amplicon. Parental 293 cells infected with HV serve as a negative control, resulting in a 605 bp amplicon. C) Equal amounts of cell lysate from passages 0-3 during HdAd production were used to transduce 293 cells. Images were captured at 24 hours post-transduction, showing expression of the fluorescent reporter mClover3 from the HdAd genome. D) Images for passages 0 and 1 were captured using an exposure time of 400 ms. Images for passages 2 and 3 were captured using an exposure time of 25 ms.

FIGS. 3A-3B. VikAD production of HdAd FLEX EGFP enables Cre dependent expression in vitro. A) Schematic of HdAd genome containing a flexed EGFP reporter, which contains two different loxP sites which allows for additional recombination events after the inversion of the intervening sequence. In the absence of Cre, only mCherry is expressed from the vector. In the presence of Cre, both mCherry and EGFP are expressed. B) 293 and 116 cells were transduced with 200 viral particles per 567 cell. Fluorescent images were captured 72 hours post-transduction.

FIGS. 4A-4D. VikAD production of HdAd FLEX EGFP enables Cre-dependent expression in a mouse Cre driver line. A) Graphical representation of injection site (Cochlear nucleus, CN) and the presynaptic terminals (Calyx of Held at MNTB region) in the contralateral MNTB in the auditory brainstem. HdAd FLEX EGFP was injected at P1 in the CN of either Math5 Cre or wild-type (wt) mice (C57BL/6J). B) Tile-scanned confocal images of (wt) (top) and Math5 Cre (bottom) mouse auditory brain stem. C,D) Magnified images of CN and MNTB C) (top) CN injected with HdAd FLEX EGFP shows that only mCherry is expressed in the absence of Cre. (bottom) Math5 Cre CN demonstrates EGFP expression in the presence of Cre. D) (top) Calyces of Held in the contralateral MNTB of wt mice. (bottom) Calyces of Held in the contralateral MNTB of Math5 Cre mice.

FIGS. 5A-5D. Co-injection of HdAd Cre and HdAd FLEX EGFP results in Cre-dependent EGFP expression in a native neuronal circuit. A) Cartoon of the auditory brainstem where HdAd Cre and HdAd FLEX EGFP were co-injected into the P1 CN of C57BL6/J mice. B) Representative images of mCherry and EGFP expression in the P9 auditory brainstem. (C, D) Magnified images of mCherry and EGFP expression from the C) CN and D) contralateral MNTB.

FIG. 6. Overview of the Vika-vox HdAd production system. A cell line expressing Vika recombinase is transfected with a linearized plasmid containing the HdAd genome. Cells are subsequently infected with a helper adenovirus that contains a packaging signal flanked by vox sites. During viral genome replication, the packaging signal in the helper plasmid is excised through the site-specific recognition of the vox sites by Vika recombinase, rendering the helper genome unable to be incorporated into progeny viral particles. The resulting virus contains only HdAd genomes.

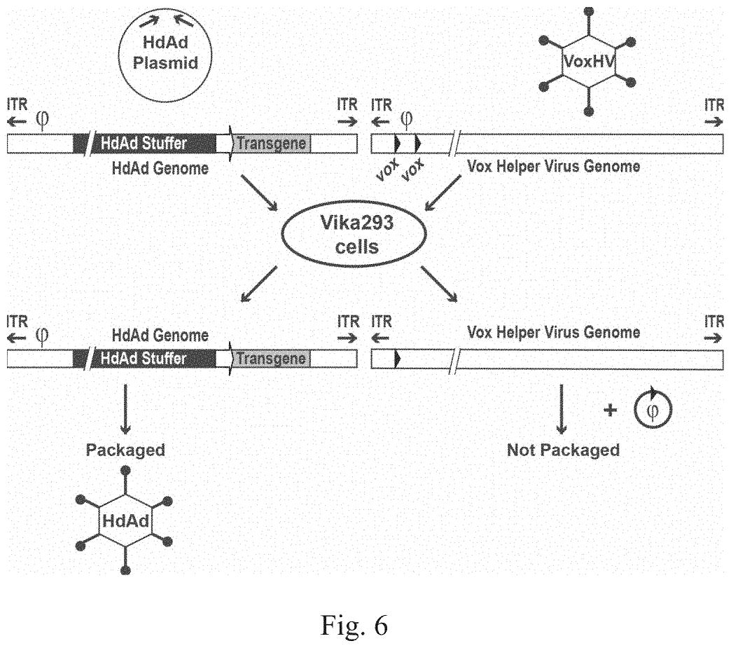

### DETAILED DESCRIPTION

The viral sequences in the HdAd genome are the inverted terminal repeats (ITRs) and packaging signal sequence (ψ), which are required for genome replication and packaging into vector particles (Palmer & Ng, 2011b). Therefore, HdAd production requires that the Ad viral proteins are supplied in trans with correct temporal regulation (Palmer & Ng, 2011b). The most successful HdAd production systems utilize a 1st generation E1-deleted Ad vector as a helper virus (HV) to mediate HdAd genome replication since expression of viral proteins follows temporal timescale that allows for high titer HdAd vector production, e.g., at least 10⁴ viral particles per cell, in producer cells expressing the Ad E1 proteins (Palmer & Ng, 2003; Umana et al., 2001) although titers from other producer cells such as those that express proteins from the E2 or E4 region in trans or express other regions in the viral genome may be employed to supply viral proteins in trans. Since HV is required to produce high titers of HdAd, strategies that utilize either Cre/loxP or Flp/FRT site-specific recombination have been designed to prevent HV genome packaging while allowing HdAd genome packaging into progeny vector particles (Palmer & Ng, 2003; Umana et al., 2001; Ng et al., 2001). These production systems use a combination of HEK293 (293) producer cells expressing the Ad E1 proteins and high levels of either Cre or Flp recombinases and a HV packaging signal (ψ) that is flanked by either loxP or FRT sites. These systems and their purification strategies allow the generation of HdAd vectors with similar titers to 1st generation Ad vectors with minor amounts of HV contamination (about 0.5-0.01%) (Palmer & Ng, 2003; Umana et al., 2001; Ng et al., 2001).

The disclosure provides for production of purified preparations of HdAd vector that contain of loxP, loxP derivative sites, FRT sites, noxP, and pox sites and other site-specific recombinase recognitions for genetic intersectional strategies that enable strategies using Cre and Flp or other site specific recombinases in vitro and in vivo.

The systems disclosed herein, e.g., VikAd, make use of Vika, a site-specific recombinase, for removal of Ad helper virus during HdAd production. This allows for the inclusion of loxP, loxP derivative sites, FRT sites, noxP, and pox sites and other site-specific recombinase recognition sites that is not possible with current Cre and FLP recombinase HdAd production systems. VikAd thus fills an important need for intersectional genetic strategies for cell-type specific expression of large genes and transgene expression cassettes that cannot be packaged into rAAV or LVV.

### Definitions

A “vector” or “construct” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest for gene therapy. Vectors include, for example, viral vectors, isolated RNA or DNA, e.g., plasmids, liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Large varieties of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

An “expression vector” is a vector comprising a region which encodes a gne product, e.g., a polypeptide, of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

A “recombinant viral vector” refers to a viral vector comprising one or more heterologous genes or sequences. Since many viral vectors exhibit size constraints associated with packaging, the heterologous genes or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying genes necessary for replication and/or encapsidation). Modified viral vectors in which a polynucleotide to be delivered is carried on the outside of the viral particle have also been described.

“Adenovirus” is used to refer to the naturally occurring wild-type virus itself or derivatives thereof. The term covers all subtypes, serotypes and pseudotypes, and both naturally occurring and recombinant forms, except where required otherwise. As used herein, the term “serotype” or “subtype” refers to an adenovirus which is identified by and distinguished from other adenoviruses based, e.g., capsid protein reactivity with defined antisera or genetic relatedness. For each example illustrated herein the description of the vector design and production describes the serotype of the capsid and 5′-3′ ITR sequences.

An “adenovirus vector” as used herein refers to an adenovirus sequence containing vector comprising a polynucleotide sequence not of adenovirus origin (i.e., a polynucleotide heterologous to adenovirus), typically a sequence of interest for the genetic transformation of a cell. In some vector constructs of this invention, the heterologous polynucleotide is flanked by one or two adenovirus inverted terminal repeat sequences (ITRs). The term adenovirus vector encompasses both viral particles and plasmids.

A “helper virus” for adenovirus refers to a virus that allows another adenovirus genome to be replicated and packaged by a mammalian cell. For example, the genome of the helper virus may be from an adenovirus type 5 of subgroup C. Numerous adenoviruses of human, non-human mammalian and avian origin are known and available from depositories such as the ATCC.

A “Chimeric virus” or “Chimeric viral particle” refers to a viral particle composed of at least one capsid protein and an encapsidated polynucleotide, which is from a different virus, serotype or subtype.

An “infectious” virus or viral particle is one that comprises a polynucleotide component, which it is capable of delivering into a cell for which the viral species is trophic. The term does not necessarily imply any replication capacity of the virus.

Tropism as used herein, is a term referring to the ability of a particular viral serotype to productively infect cells of differing phenotypes or organs to deliver their genomic information to the nucleus.

A “gene” refers to a polynucleotide containing at least one open reading frame that is capable of encoding a particular protein after being transcribed and translated.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art.

By “transgene” is meant any piece of a nucleic acid molecule (for example, DNA) which is inserted by artifice into a cell either transiently or permanently, and becomes part of the organism if integrated into the genome or maintained extrachromosomally. Such a transgene includes at least a portion of an open reading frame of a gene which is partly or entirely heterologous (i.e., foreign) to the transgenic organism, or may represent an open reading frame or a portion thereof of a gene homologous to an endogenous gene of the organism, which portion optionally encodes a polypeptide with substantially the same activity as the corresponding full length polypeptide, e.g., wild-type polypeptide, or at least one activity of the corresponding full length polypeptide.

By “transgenic cell” is meant a cell containing a transgene. For example, a stem cell transformed with a vector containing an expression cassette can be used to produce a population of cells having altered phenotypic characteristics. A “recombinant cell” is one which has been genetically modified, e.g., by insertion, deletion or replacement of sequences in a nonrecombinant cell by genetic engineering.

The term “wild-type” or “native” refers to a gene or gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “transduction” denotes the delivery of a polynucleotide to a recipient cell either in vivo or in vitro, via a viral vector and preferably via a replication-defective viral vector.

“Gene expression” or “expression” refers to the process of gene transcription, translation, and post-translational modification.

A “detectable marker gene” is a gene that allows cells carrying the gene to be specifically detected (e.g., distinguished from cells which do not carry the marker gene). A large variety of such marker genes are known in the art.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. A variety of positive and negative selectable markers are known in the art, some of which are described below.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

By “DNA” is meant a polymeric form of deoxyribonucleotides (adenine, guanine, thymine, or cytosine) in double-stranded or single-stranded form found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence complementary to the mRNA). The term captures molecules that include the four bases adenine, guanine, thymine, or cytosine, as well as molecules that include base analogues which are known in the art.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

“Heterologous” means derived from a genotypically distinct entity from that of the rest of the entity to which it is compared. For example, a polynucleotide introduced by genetic engineering techniques into a different cell type is a heterologous polynucleotide (and, when expressed, can encode a heterologous polypeptide). Similarly, a TRS or promoter that is removed from its native coding sequence and operably linked to a different coding sequence is a heterologous TRS or promoter. The term “heterologous” as it relates to nucleic acid sequences such as gene sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature, i.e., a heterologous promoter. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention.

The term “isolated” when used in relation to a nucleic acid, peptide, polypeptide or virus refers to a nucleic acid sequence, peptide, polypeptide or virus that is identified and separated from at least one contaminant nucleic acid, polypeptide or other biological component with which it is ordinarily associated in its natural source. Isolated nucleic acid, peptide, polypeptide or virus is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded).

An “isolated” plasmid, virus, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture.

A “transcriptional regulatory sequence” or “TRS,” as used herein, refers to a genomic region that controls the transcription of a gene or coding sequence to which it is operably linked. Transcriptional regulatory sequences of use in the present invention generally include at least one transcriptional promoter and may also include one or more enhancers and/or terminators of transcription. The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′ direction) coding sequence. Promoters include native adenovirus promoters, as well as heterologous promoters. By “enhancer” is meant a nucleic acid sequence that, when positioned proximate to a promoter, confers increased transcription activity relative to the transcription activity resulting from the promoter in the absence of the enhancer domain.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. The term “control elements” refers collectively to promoter regions, polyadenylation signals, transcription termination sequences, upstream regulatory domains, origins of replication, internal ribosome entry sites (“IRES”), enhancers, splice junctions, and the like, which collectively provide for the replication, transcription, post-transcriptional processing and translation of a coding sequence in a recipient cell. Not all of these control elements need always be present so long as the selected coding sequence is capable of being replicated, transcribed and translated in an appropriate host cell.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences,” are specific sequences that tend to disrupt read-through transcription by RNA polymerase, presumably by causing the RNA polymerase molecule to stop and/or disengage from the DNA being transcribed. Typical examples of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and more preferably not less than 19 matches out of 20 possible base pair matches (95°/o).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more preferably homologous if their amino acids are greater than or equal to 50% identical when optimally aligned using the ALIGN program.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

“Packaging” as used herein refers to a series of subcellular events that results in the assembly and encapsidation of a viral vector. Thus, when a suitable vector is introduced into a packaging cell line under appropriate conditions, it can be assembled into a viral particle. Functions associated with packaging of viral vectors are described herein and in the art.

“Host cells,” “cell lines,” “cell cultures,” “packaging cell line” and other such terms denote higher eukaryotic cells, e.g., mammalian cells, such human cells, useful in the present invention. These cells can be used as recipients for recombinant vectors, viruses or other transfer polynucleotides, and include the progeny of the original cell that was transduced. It is understood that the progeny of a single cell may not necessarily be completely identical (in morphology or in genomic complement) to the original parent cell.

A “therapeutic gene,” “prophylactic gene,” “target polynucleotide,” “transgene,” “gene of interest” and the like generally refer to a gene or genes to be transferred using a vector. Typically, in the context of the present invention, such genes are located within the HdAd vector (which vector is flanked by inverted terminal repeat (ITR) regions and thus can be replicated and encapsidated into adenovirus particles). Target polynucleotides can be used in this invention to generate HdAd vectors for a number of different applications. Such polynucleotides include, but are not limited to: (i) polynucleotides encoding proteins useful in other forms of gene therapy to relieve deficiencies caused by missing, defective or sub optimal levels of a structural protein or enzyme; (ii) polynucleotides that are transcribed into anti-sense molecules; (iii) polynucleotides that are transcribed into decoys that bind transcription or translation factors; (iv) polynucleotides that encode cellular modulators such as cytokines; (v) polynucleotides that can make recipient cells susceptible to specific drugs, such as the herpes virus thymidine kinase gene; and (vi) polynucleotides for cancer therapy, such as E1A tumor suppressor genes or p53 tumor suppressor genes for the treatment of various cancers. To effect expression of the transgene in a recipient host cell, it is operably linked to a promoter, either its own or a heterologous promoter. A large number of suitable promoters are known in the art, the choice of which depends on the desired level of expression of the target polynucleotide; whether one wants constitutive expression, inducible expression, cell-specific or tissue-specific expression, etc. The HdAd vector may also contain a selectable marker.

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

“Genetic alteration” refers to a process wherein a genetic element is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a DNA or RNA virus or viral vector. The genetic element may be introduced into a chromosome or mini-chromosome in the cell; but any alteration that changes the phenotype and/or genotype of the cell and its progeny is included in this term.

“Gene targeting” is a genetic technique that uses homologous recombination to change an endogenous gene, e.g., delete the gene, remove exons, add a gene or alter the splice donor and receptor sequence, add a gene, or change one or more bases in the DNA.

“Gene editing” is a type of genetic engineering in which DNA is inserted, deleted or replaced in the genome of an organism using a recombinant nuclease, e.g., a heterologous nuclease.

“Gene correction template” is the exogenous template that is used to introduce DNA into, delete DNA from or otherwise replace or alter DNA in the genome of an organism.

A cell is said to be “stably” altered, transduced or transformed with a genetic sequence if the sequence is available to perform its function during extended culture of the cell in vitro. In some examples, such a cell is “inheritably” altered in that a genetic alteration is introduced which is also inheritable by progeny of the altered cell.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component. Polypeptides, when discussed in the context of gene therapy and compositions therefor, refer to the respective intact polypeptide, or any fragment or genetically engineered derivative thereof, that retains the desired biochemical function of the intact protein. Similarly, references to genes for use in gene therapy, include polynucleotides encoding the intact polypeptide or any fragment or genetically engineered derivative possessing the desired biochemical function.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, about 90%, about 95%, and about 99%. For example, the object species is purified to essential homogeneity (contaminant species cannot be detected in the composition by conventional detection methods) wherein the composition consists essentially of a single macromolecular species. For example, a preparation of virus is said to be “substantially free” of helper virus if the ratio of infectious HdAd particles to infectious HV particles is at least about 10²:1; e.g., at least about 10³:1, including at least about 10⁴:1, or at least about 10⁵:1.

A preparation of HdAd is said to be “substantially free” of helper virus if the ratio of infectious HdAd particles to infectious helper virus particles is at least about 10²:1; e.g., at least about 10⁴:1, including at least about 10⁶:1 or at least about 10⁸:1. Preparations may also be free of equivalent amounts of helper virus proteins (i.e., proteins as would be present as a result of such a level of helper virus if the helper virus particle impurities noted above were present in disrupted form). Viral and/or cellular protein contamination may generally be observed as the presence of Coomassie staining bands on SDS gels.

“Efficiency” when used in describing viral production, replication or packaging refers to useful properties of the method: in particular, the growth rate and the number of virus particles produced per cell. “High efficiency” production indicates production of at least 100 viral particles per cell; e.g., at least about 10,000 or at least about 100,000 particles per cell, over the course of the culture period specified.

An “individual” or “subject” treated in accordance with this invention refers to vertebrates, particularly members of a mammalian species, and includes but is not limited to domestic animals, sports animals, and primates, including humans.

The term “prophylactically effective amount” is meant to refer to the amount necessary to, in the case of infectious agents, prevent an individual from developing an infection, and in the case of diseases, prevent an individual from developing a disease.

The term “therapeutically effective amount” is meant to refer to the amount necessary to, in the case of infectious agents, reduce the level of infection in an infected individual in order to reduce symptoms or eliminate the infection, and in the case of diseases, to reduce symptoms or cure the individual.

“Inducing an immune response against an immunogen” is meant to refer to induction of an immune response in a naïve individual and induction of an immune response in an individual previously exposed to an immunogen wherein the immune response against the immunogen is enhanced.

“Treatment” of an individual or a cell is any type of intervention in an attempt to alter the natural course of the individual or cell at the time the treatment is initiated, e.g., eliciting a prophylactic, curative or other beneficial effect in the individual. For example, treatment of an individual may be undertaken to decrease or limit the pathology caused by any pathological condition, including (but not limited to) an inherited or induced genetic deficiency, infection by a viral, bacterial, or parasitic organism, a neoplastic or aplastic condition, or an immune system dysfunction such as autoimmunity or immunosuppression. Treatment includes (but is not limited to) administration of a composition, such as a pharmaceutical composition, and administration of compatible cells that have been treated with a composition. Treatment may be performed either prophylactically or therapeutically; that is, either prior or subsequent to the initiation of a pathologic event or contact with an etiologic agent.

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like.

By “derived from” is meant that a nucleic acid molecule was either made or designed from a parent nucleic acid molecule, the derivative retaining substantially the same functional features of the parent nucleic acid molecule, e.g., encoding a gene product with substantially the same activity as the gene product encoded by the parent nucleic acid molecule from which it was made or designed.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means, or in relation a cell refers to a cell which was isolated and subsequently introduced to other cells or to an organism by artificial or natural means. An exogenous nucleic acid may be from a different organism or cell, or it may be one or more additional copies of a nucleic acid which occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

### Exemplary System for Producing Helper Dependent Adenovirus

Recombinant viral vectors have become integral tools for basic in vivo research applications. Helper-dependent adenoviral (HdAd) vectors are safe viral vectors with a large packaging capacity of ˜36 kb of DNA that mediate long-term expression without toxicity. The large carrying capacity of HdAd enables basic research or clinical applications requiring the delivery of large genes or multiple transgenes which cannot be packaged into other widely-used viral vectors. Currently, common HdAd production systems use a helper Ad virus (HV) with a packaging signal (W) that is flanked by either loxP or FRT sites which is excised in producer cells expressing Cre or Flp recombinases to prevent HV packaging. However, these production systems prevent the use of HdAd vectors for genetic strategies that rely on Cre or Flp recombination for cell-type specific expression. To overcome these limitations, we developed a new production system, which is based on novel producer cells expressing the Vika recombinase and a novel HV that contains a ψ flanked by vox sites. The availability of this production system will greatly expand the utility and flexibility of HdAd vectors for use in basic research applications to monitor and manipulate cellular activity with increased specificity.

### Exemplary Methods to Prepare a Vika Expressing Cell Line, HdAd and Helper Virus Genome

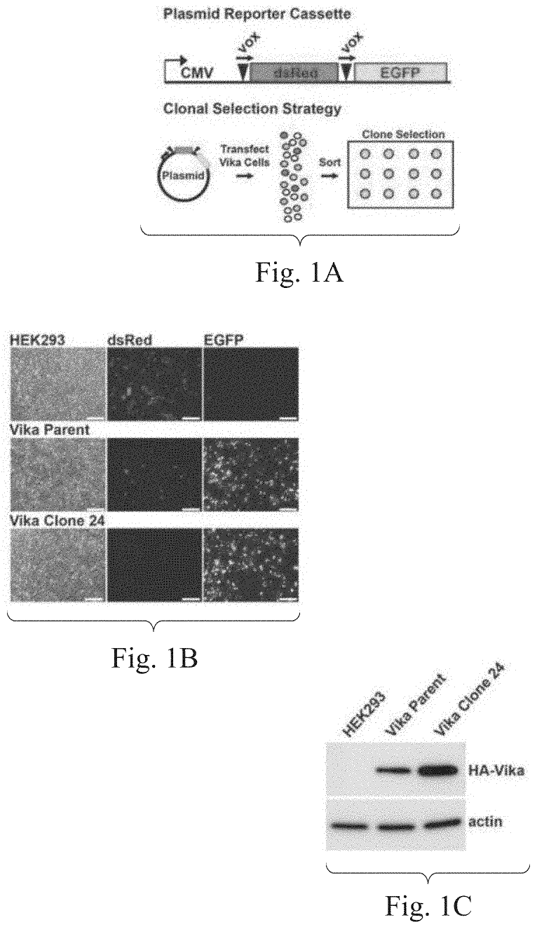

In one embodiment, the coding sequence for the Vika enzyme was synthesized using codon selection optimized for expression in human cells, e.g., ATGCTAGCCACCATGTATCCCTACGACGTGCCCGACTACGCCAAGAAA AAGCGGAAAGTGACCGATCTGACCCCATTTCCTCCTCTGGAACACCTG GAACCTGACGAGTTCGCCGATCTCGTGCGGAAGGCCATCAAGAGAGAT CCTCAGGCTGGCGCCCATCCTGCCATCCAATCTGCCATCAGCCACTTC CAGGATGAGTTCGTGCGGAGACAAGGCGAATGGCAGCCTGCCACACT GCAGAGACTGAGAAACGCCTGGAATGTGTTTGTGCGGTGGTGCACCCA CCAGGGCATTCCAGCATTGCCTGCCAGACACCAGGACGTGGAAAGATA CCTGATCGAGCGGCGGAACGAGCTGCACAGAAACACCCTGAAAGTGC ACCTGTGGGCCATCGGCAAGACCCACGTTATCAGCGGCCTGCCTAATC CATGCGCTCACAGATACGTGAAAGCCCAGATGGCCCAGATCACACACC AGAAAGTGCGCGAGAGAGAGCGGATTGAACAGGCCCCTGCCTTCAGA GAGTCCGACCTGGATAGACTGACCGAGCTTTGGAGCGCCACCAGAAG CGTTACCCAGCAGAGGGACCTGATGATCGTGTCCCTGGCCTACGAGAC ACTGCTGCGGAAGAACAATCTGGAACAGATGAAAGTGGGCGACATCGA GTTCTGCCAGGATGGCTCTGCCCTGATCACAATCCCCTTCAGCAAGAC CAACCACAGCGGCAGAGATGACGTGCGGTGGATCTCTCCTCAGGTGG CCAATCAGGTGCACGCCTATCTGCAGCTGCCCAACATCGATGCCGATC CTCAGTGCTTCCTGCTGCAGAGAGTGAAGAGAAGCGGCAAGGCCCTG AATCCTGAGAGCCACAATACCCTGAACGGCCACCATCCTGTGTCCGAG AAGCTGATCTCCCGGGTGTTCGAGAGAGCTTGGAGAGCCCTGAACCAC GAGACAGGCCCTAGATACACAGGCCACAGCGCTAGAGTTGGCGCCGC TCAAGATCTGCTGCAAGAGGGCTACAGCACCCTGCAAGTTATGCAAGC CGGCGGATGGTCCAGCGAGAAGATGGTGCTGAGATACGGCAGACATC TGCACGCCCACACAAGCGCCATGGCTCAGAAGAGAAGGCAGCGGAGC AGAGTGGAAGAGTTCAGCCTCTGA (SEQ ID NO: 14). Codon optimized Vika recombinase was synthesized with an N-terminal hemagglutinin (HA) epitope tag (YPYDVPDYA; SEQ ID NO:2) and nuclear localization signal (KKKRKV; SEQ ID NO:3). This sequence was cloned into a standard lentiviral cloning vector plasmid pFIV3.2 CMV IRES neo provided by The University of Iowa Viral Vector Core (Cat #G0752). This plasmid was used to produce lentivirus according to standard protocols. The resulting lentivirus was named FIV3.2 CMV HA-NLS-Vika IRES neo. This lentivirus was used to transduce 293 cells and transduced cells were selected by culture in growth medium containing the antibiotic G418. The resulting pooled population cell line was named HA-NLS-Vika 293. Vika enzymatic activity, e.g., the ability of Vika to mediated site-specific excision of DNA between vox sites, was confirmed by transfection of a fluorescent reporter plasmid (FIG. 1).

Clonal populations of Vika cells were used to identify individual clones with high levels of Vika expression and activity (FIG. 1). Parental Vika cells were transfected with pCMV-vox-dsRed-vox-EGFP followed by live single-cell FACS to select for individual cells that expressed EGFP but not dsRed and clones with high levels of EGFP and undetectable dsRed expression were amplified. Clones in which the reporter plasmid was found to have integrated into the chromosomal DNA were excluded from further analysis. Based on our screening we identified a clone, Vika24 which showed almost no dsRed expression compared to the pooled population. This clone was chosen for subsequent HdAd production and is renamed as Vika293 cells.

An exemplary starting material to create the vox helper virus is the plasmid pNG163-R2 which contains the human adenoviral type 5 genome (AC_00008.1) that is E1-deleted and contains loxP sites flanking the viral packaging signal (ψ) (Parks, Chen et al. 1996, Palmer and Ng 2003). Each LoxP recognition site is 34 base pairs (bp) and consists of two 13 bp palindromic repeats. The loxP recognition sequence is:

(SEQ ID NO: 30)
ATAACTTCGTATA-GCATACAT-
TATACGAAGTTAT.

The palindromic repeats are indicated by underline. To construct the vox helper plasmid, the region of DNA containing the loxP sites and the ψ was removed. This region was then replaced with synthetic DNA containing vox sites flanking the ψ. Vox recognition sites are 34 bp and consist of two 13 bp imperfect palindromic repeats. A vox recognition sequence is:

(SEQ ID NO: 31)
AATAGGTCTGAGA-ACGCCCAT-
TCTCAGACGTATT

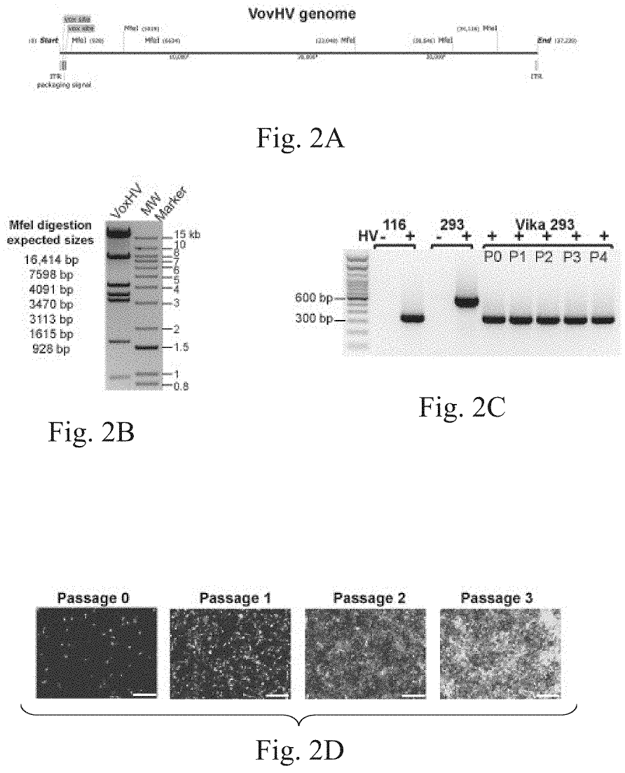

The palindromic repeats are indicated by underline and nucleotides (Karimova, Abi-Ghanem et al. 2013). The resulting plasmid was sequenced to confirm replacement of loxP sites with vox sites and was named pVoxHV (FIG. 2)

pVovHV was used to transfect HEK-293 (293) cells (ATCC CRL-1573) to produce and amplify vox helper virus (VoxHV) according to standard protocols (Palmer and Ng 2011) (FIG. 2). Virus was purified based on density by sequential centrifugation in cesium chloride. The infectivity of the VoxHV was verified by infection of 293 cells and quantified by digital drop PCR, capsid protein expression, and plaque assay.

A helper adenovirus that has been genetically modified to have vox sites flanking the packaging signal and a producer cell line that stably expresses high levels of catalytically active Vika enzyme are employed for the production of HdAds containing site-specific recombination sites including but not limited to loxP, loxP derivative sites, FRT sites, noxP, pox sites or other site-specific recomibinase recognition sites. For example the use of, VikAd, enables the production of HdAd vectors that utilize Cre, Flp, Cre Derivative and other site-specific recombinase excision for transgene expression in vitro and in vivo.

VikAd was used to produce an HdAd vector that contains loxP sites in its genome (FIG. 2). To create pHdAd FLEx EGFP, the FLEX EGFP sequence from Addgene plasmid 59331 (pAAV-CAG-FLEX-EGFP was a gift from Ian Wickersham; http://n2t.net/addegen:59931 RRID:Addgene 5933) was cloned into our standard syn BGH polyA shuttle vector which uses the 470 bp human synapsin promoter to drive neurospecific expression (Lubbert, Goral et al. 2017). The syn FLEx EGFP BGH polyA cassette was then cloned into the HdAd plasmid C4HSU syn mCherry (Lubbert, Goral et al. 2017, Lubbert, Goral et al. 2019) to create pHdAd syn FLEx EGFP syn mcherry, which contains two separate transgene cassettes for reporter expression (FIG. 2). To test the efficiency of the VikAD production system, a HdAd CMV mClover3 vector was amplified. Crude lysates were examined at each passage of HdAd production for both excision of VoxHV and HdAd amplification. To evaluate excision of the ψ from VoxHV in Vika293 cells, standard non-quantitative PCR using 30 cycles of amplification and primers that flank the ψ from VoxHV was performed (FIG. 2B). As a positive control for excision, 116 cells were infected with the loxP HV(Palmer and Ng 2003). As a negative control for excision, 293 cells were infected with the voxHV (FIG. 2). Based on our PCR excision assay we were unable to detect VoxHV genome with the ψ intact at any passage of HdAd amplification

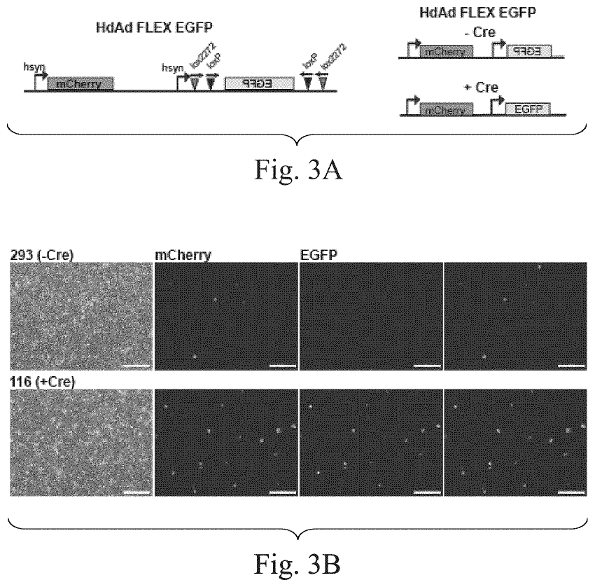

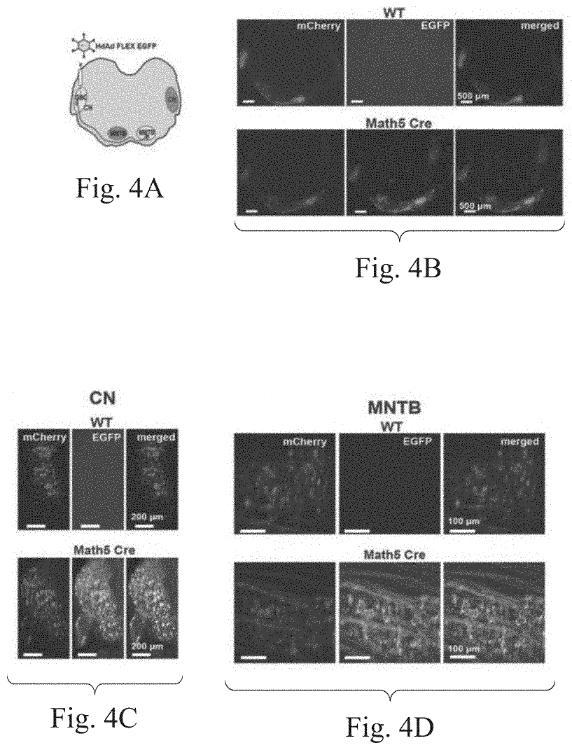

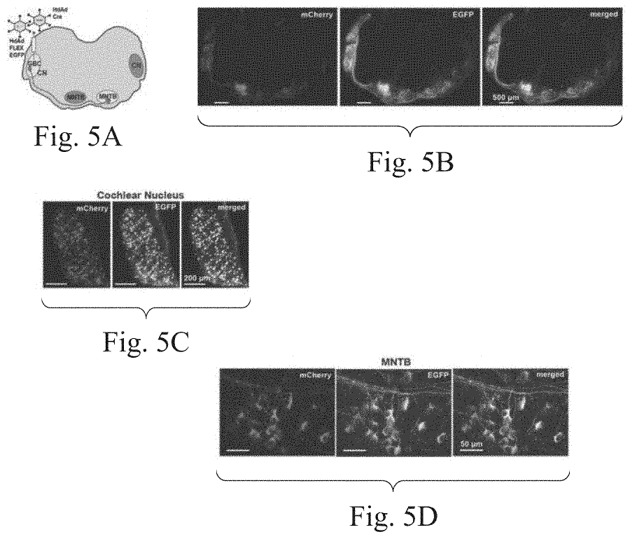

To determine if Cre mediated excision led to EGFP expression in HdAd FLEx EGFP in an in vitro context, 116 cells (Palmer and Ng 2003) (Cre+) or 293 cells (Cre−) were infected. Although the hSyn promoter is a neurospecific promoter (Kugler, Meyn et al. 2001), the hsyn promoter has low levels of activity in 293 cells and their derivatives as 293 cells are related to neuronal cells (Shaw, Morse et al. 2002). Analysis of the 116 and 293 cells 72 hrs after infection (FIG. 3) revealed that 293 cells only expressed mCherry, however 116 cells which were mCherry positive were also EGFP positive, indicating successful inversion of the FLEXed EGFP sequence. To test compatibility with a mouse Cre driver line in a native neuronal context, HdAd FLEX EGFP was injected into the P1 mouse cochlear nucleus (CN) (Chen, Cooper et al. 2013) of the Math5 mouse Cre driver line. Math5 (Atoh7) is a transcription factor that leads to selective labeling of the Globular bushy cells (GBCs) in the CN (Saul, Brzezinski et al. 2008, Xu, Wu et al. 2016). Subsequent analysis of the P9 auditory brainstem (FIG. 4) revealed EGFP expression in the CN, the GBC axons and the calyces of Held, large glutamatergic presynaptic terminals which arise from the GBCs axon. Finally, to test if co-delivery of HdAd FLEC EGFP with an HdAd Cre vector leads to efficient EGFP expression, we co-injected an HdAd Cre which contains a hsyn mCherry reporter (Lubbert, Goral et al. 2017) and HdAd FLEx EGFP into the P1 mouse CN (FIG. 5). Analysis of the P9 auditory brainstem revealed EGFP expression that co-localized with mCherry expression in the CN and axons that projected from the CN. In particular, the calyx of Held were co-labeled with EGFP and mCherry in the contra lateral MNTB from the injection site

### Exemplary Vika Sequences, Host Cells and Adenovirus Helper Genomes

Exemplary Vika sequences for expression in a eukaryotic host cell such as a mammalian cell including human or non-human primate cells, canine cells, feline cells, bovine cells, equine cells, rodent cells, e.g., mouse or rat cells, include but are not limited to:

(SEQ ID NO: 20)
mtdltpfppl ehlepdefad Ivrkaikrdp
qagahpaiqs aishfqdefv rrqgewqpat
Iqrlrnawnv fvrwcthqgi palparhqdv
erylierrne ihrntlkvhl waigkthvis
glpnpcahry vkaqmaqith qkvrererie
qapafresdi drltelwsat rsvtqqrdlm
ivslayetll rknnleqmkv gdiefcqdgs
alitipfskt nhsgrddvrw ispqvanqvh
aylqlpnida dpqcfllqrv krsgkalnpe
shntinghhp vseklisrvf erawralnhe
tgprytghsa rvgaaqdllq egystlqvmq
aggwssekmv Irygrhlhah tsamaqkrr
r,
(SEQ ID NO: 21)
makttvkitp tkiksskpkd keynlfdgdg
lrlrikpngs khwiinyyrp snrkrarlsl
gkypdlslan arkmtleake llaqgidpqe
erkrhqleqk anhehtfinv ttkwfeikkd
dvtpdyavdi wrslelhifp qlsdvpvrdi
tapqviellk pieakgslet vkrlaqrlne
vmnyatncgl vqanpltgik aafkkpkken
maaltpaelp elmgalanas ikrttrclie
wqlhtmtrps easgarweei dwdekvwtip
permkkrreh riplteqmle Ilevikpisg
hrefifpsdr dpkkpcnsqt anmalkrmgf
agrlvshglr slasttlneq gfepdlieaa
lahaddnqvr saynrtdyle rrrpmmcwws
ghieeaakgs Isvtgmkqlk vinvd,
(SEQ ID NO: 22)
mtdltpfppl ehlepdefad Ivrkaikrdp
qagahpaiqs aishfqdefv rrqgewqpat
Iqrlrnawnv fvrwcthqgi palparhqdv
erylierrne Ihrntlkvhl waigkthvis
glpnpcahry vkaqmaqith qkvrererie
qapafresdi drltelwsat rsvtqqrdlm
ivslayetll rknnleqmkv gdiefcqdgs
alitipfskt nhsgrddvrw ispqvanqvh
aylqlpnida dpqcfllqrv krsgkalnpe
shntinghhp vseklisrvf erawralnhe
tgprytghsa rvgaaqdllq egystlqvmq
aggwssekmv Irygrhlhah tsamaqkrrq
r
(SEQ ID NO: 23)
mpekspflre vcehmrlsgy sirteksyly
wirsyifyhe krhpmqmgse eviqflsyia
nsrnvaintq kvalnalvyl yqaflkielg
elgfrhatkq rqlpvvitpn evkqvlshln
gkeklivell ygsglrisec lrlrvqdidl
snlsitvrdg kgkkdrqtll skqcvdvlkl
lleqaeqvqi adnkhgigps lpyaihkkyp
nafrklawmf ifpspsishs qynsdhpsrh
hlhssvirka Isravaktti nkkvtchtfr
hsfathllqa grdirsvqel Ighndvkttq
iythvigqhy agttspldnl n,
(SEQ ID NO: 24)
mpekspflre vcehmrlsgy sirteksyly
wirsyifvhe krhpmqmgse eviqflsyia
nsrnvaintq kvalnalvyl yqtflkielg
elgfrhatkq rqlpvvitpn evkqvlshln
gkeklivell ygsglrisec lrlrvqdidl
snlsitvrdg kgkkdrqtll skqcvdvlki
lieqaeqvqi adnkhgigps lpyaihkkyp
nafrklawmf ifpspsishs qynndhpsrh
hlhssvirka Isravaktti nkkvtchtfr
hsfathllqa grdirsvqel Ighndvkttq
iythvigqhy agttspldnl n,
(SEQ ID NO: 25)
msesaiplpn glqsplnrfy eylrsekgls
lhtqrnykqq letmaqhlvt mglkdwtqvd
aawvrqlask gmrdgmkass latrlsalrs
ffdflilrge Itanpakgvs aprkkrplpk
nldvdevgql levneddpla irdrammelm
ygaglrlaem vsvdvrdvsl ssgeirvvgk
gdkerkvpfa gmaeewvakw Ikvrgvlant
depalfvskl gvrishrnvq krmaewgqkq
svashisphk Irhsfathml essnnlravq
ellghenist tqiythldfq hladvydqah
prakkkesd,

or a polypeptide with at least 80%, 85%, 90%, 92%, 95%, 96%, 97%, 98% or 99% amino acid sequence identity thereto, or mtdltpfppl ehlepdefad lvrkaikrdp gagahpaigs aishfqdefv rrggewgpat lqrlrnawnv fvrwcthqgi palparhqdv erylierrne lhrntlkvhl waigkthvis glpnpcahry vkagmagith qkvrererie gapafresdl drltelwsat rsvtggrdlm ivslayetll rknnlegmkv gdiefcgdgs alitipfskt nhsgrddvrw ispgvangvh aylglpnida dpqcfllqry krsgkalnpe shntinghhp vseklisrvf erawralnhe tgprytghsa rvgaaqdllq egystlqvmq aggwssekmv lrygrhlhah tsamaqkrrq r (SEQ ID NO:1), or a polypeptide with at least 80%, 85%, 90%, 92%, 95%, 96%, 97%, 98% or 99% amino acid sequence identity thereto.

The Vika recombinase may be one from the genus Vibrio, e.g., Vibrio coraiiilyticus, V europaeus, V. tubiashii, V. ouch, V parahaemolyticus, V. navarrensis, or V. ponticus.

In one embodiment, the host cell may recombinantly express one or more adenovirus proteins that may complement, or augment, genes in the adenovirus helper genome. For example, the host cell may express one or more of E1A, E1B, pIX, E2B, IVa2, E2A, E3, E4, L1, L2, L3, L4 or L5 to complement a disabled gene in the adenovirus helper genome, e.g., a genome that has a disabling mutation in one or more of E1A, E1 B, pIX, E2B, 1Va2, E2A, E3, E4, LI, L2, L3, L4 or L5. In one embodiment, the host cell is a human cell. In one embodiment, the human cell expresses E1A, E1 B, or both, and the adenovirus helper genome has a disabling mutation in Elk E1B, or both.

An adenovirus helper genome vector having at least one adenovirus ITR and a packaging signal flanked by vox sites may be prepared from any adenovirus serotypes, e.g., from any one of subgroups A to G. For instance, the helper genome may be from subgroup B, Ad 3, Ad7, Ad11, Ad 14, Ad35 or Group D. The adenovirus helper genome vector may include sequences encoding all adenovirus proteins or fewer than all adenovirus proteins, e.g., the adenovirus helper genome vector may have fewer than about 35,000 bp of the adenovirus genome.

An HdAd vector having two adenovirus ITRs and an adenovirus packaging signal, which optionally includes at least one coding region for an adenovirus protein. In one embodiment, the HdAd vector does not encode any adenovirus protein. The adenovirus sequences in the HdAd vector may be from any adenovirus serotype.

The invention will be further described by the following non-liming example.

### Example 1

Recombinant viral vectors have become integral tools for basic in vivo research applications. Helper-dependent adenoviral (HdAd) vectors have a large packaging capacity of about 36 kb of DNA that mediate long-term transgene expression without toxicity in vitro and in vivo. The large carrying capacity of HdAd enables basic research or clinical applications requiring the delivery of large genes or multiple transgenes which cannot be packaged into other widely-used viral vectors. Currently, common HdAd production systems use a helper Ad virus (HV) with a packaging signal (ψ) that is flanked by either loxP or FRT sites, which is excised in producer cells expressing Cre or Flp recombinases to prevent HV packaging. However, these production systems prevent the use of HdAd vectors for genetic strategies that rely on Cre or Flp recombination for cell-type specific expression. To overcome these limitations, a VikAD production system was prepared, which is based on producer cells expressing the Vika recombinase and HV that contains a‘I’ flanked by vox sites. The availability of this production system expands the utility and flexibility of HdAd vectors for use in research applications to monitor and manipulate cellular activity with increased specificity.

### Materials and Methods

### Ethical Approval

All mice were used in accordance with animal welfare laws approved by the Institutional Committee for Care and Use of Animals at the University of Iowa (0021952). Animals were housed with 12 hours light/dark cycle and ab libitum food/water supply. Mice of both sexes were used for all experiments. C57BL/6J mice (wild-type, wt) were obtained from Jax labs and bred inhouse. Atoh7 (Math5) Cre lines were generated using by the University of Iowa Genome Editing Facility.

Generation of Math5 Cre mice

C57BL/6J mice were purchased from Jackson Labs (000664; Bar Harbor, ME). Male mice older than 8 weeks were used to breed with 3-5 week old super-ovulated females to produce zygotes for pronuclear injection. Female ICR (Envigo; Hsc:ICR(CD-1)) mice were used as recipients for embryo transfer. All animals were maintained in a climate-controlled environment at 25° C. and a 12/12 light/dark cycle. Animal care and procedures conformed to the standards of the Institutional Animal Care and Use Committee of the Office of Animal Resources at the University of Iowa.

### Preparation of Cas9 RNPs and the injection mix

Chemically modified CRISPR-Cas9 crRNAs and CRISPR-Cas9 tracrRNA were purchased from Integrated DNA Technologies (IDT, Coralville, Iowa, USA, Alt-R® CRISPR-Cas9 crRNA; Alt-R® CRISPR-Cas9 tracrRNA (Cat #1072532)). The crRNAs and tracrRNA were suspended in TIOE0.1 and combined to 1 μg/ul (about 29.5 uM) final concentration in a 1:2 (μg:μg) ratio. The RNAs were heated at 98° C. for 2 minutes, then allowed to cool slowly to 20° C. in a thermal cycler. The annealed cr:tracrRNAs were aliquoted to single-use tubes and stored at −80° C. Cas9 nuclease was also purchased from IDT (Alt-R® S.p. HiFi Cas9 Nuclease). Individual cr:tracr:Cas9 ribonucleoprotein (RNP) complexes were made by combining Cas9 protein and cr:tracrRNA in T10E0.1 (final concentrations: 300 ng/μl (about 1.9 μM) Cas9 protein and 200 ng/μl (about 5.9 μM) cr:tracrRNA. The Cas9 protein and annealed RNAs were incubated at 37° C. for 10 minutes. The RNP complexes were combined with single-stranded repair template and incubated an additional 5 minutes at 37° C. The concentrations in the injection mix were 60 ng/μl (about 0.37 μM) Cas9 protein, 20 ng/μl (about 0.59 μM) each cr:tracrRNA and 20 ng/μl single stranded repair template.

### Collection of Embryos and Injection

Pronuclear-stage embryos were collected using standard methods (Pinkert, (2002). Embryos were collected in KSOM media (Millipore; MR101D) and washed 3 times to remove cumulous cells. Cas9 RNPs and single stranded repair template were injected into the pronuclei of the collected zygotes and incubated in KSOM with amino acids at 37° C. under 5% CO₂ until all zygotes were injected (Miura et al., 2018). Fifteen to 25 embryos were immediately implanted into the oviducts of pseudo-pregnant ICR females.

### Cell Lines

293 human embryonic kidney (293) cells and HeLa cells were maintained in Dulbecco's Modified Eagle Medium (DMEM) containing 10% fetal bovine serum (FBS), 1 mM sodium pyruvate, and 100 U/ml penicillin and streptomycin. 293 cells contain the left hand 4344 bp of the adenovirus serotype 5 genome, which expresses the viral E1A and E1B genes necessary for the replication of E1-deleted adenoviral vectors. Vika293 cells were maintained in the same medium containing 1 mg/ml G418 (IBI Scientific, Dubuque, Iowa, USA). 116 cells (kind gift from Philip Ng, Baylor College of Medicine, ref.) were maintained in Modified Eagle Medium (MEM) containing 10% FBS, 100 U/ml penicillin and streptomycin, and 100 μg/ml hygromycin (Life Technologies, Carlsbad, Calif., USA).

### Vika293 Creation

Codon optimized Vika recombinase was synthesized with an N-terminal hemagglutinin (HA) epitope tag (YPYDVPDYA; SEQ ID NO. 2) and nuclear localization signal (KKKRKV; SEQ ID NO:3) (Thermo Fisher Scientific, Waltham, Mass., USA). The synthesized fragment was cloned into the pFIV3.2CMVmcwtlRESneomycin lentiviral vector (The University of Iowa Viral Vector Core Facility, Iowa City, IA, USA). Lentivirus was produced by The University of Iowa Viral Vector Core. 293 HEK cells were transduced with Vika lentivirus and transduced cells were selected by growth in medium containing 1 mg/ml G418. To establish clonal populations exhibiting high levels of Vika activity, Vika cells were transfected with a plasmid containing a CMV-vox-dsRed-vox-EGFP reporter cassette. Twenty-four hours post-transfection cells were subjected to live cell fluorescence activated cell sorting (FACS), gating on cells that were EGFP-positive and dsRed-negative (UIOWA flow Cytometry Core). Single cells were sorted into poly-L-lysine coated 96-well plates (Corning Life Sciences, Tewksbury, MA, USA). Clones were expanded and assessed for excision activity and expression by reporter plasmid transfection and Western blot, respectively.

### Vox Helper Virus

pVoxHV plasmid was created by removing the loxP sites from the pNG163-R2 (kind gift from Philip Ng, Baylor College of Medicine, ref.) HV plasmid and replacing them with the 34 bp vox sites in a parallel orientation (5′-AATAGGTCTGAGAACGCCCATTCTCAGACGTATT-3; SEQ ID NO. 4). CRISPR/Cas9-mediated cleavage of the regions containing the loxP sites in pNG163-R2 was employed using the following sgRNA sequences: upstream of loxP-GTGTGATGTTGCAAGTGTGG-3′ (SEQ ID NO:5), downstream of loxP 5′-AATGCTTCCATCAAACGAGT-3′ (SEQ ID NO:6) sgRNA synthesis was carried out using the EnGen sgRNA Synthesis Kit and sgRNAs were purified using the Monarch RNA Clean Up Kit (New England Biolabs (NEB), Ipswich, Mass., USA). Plasmid DNA was digested in vitro with Cas9 nuclease from S. Pvogenes (NEB, Ipswich, Mass., USA). Reactions were treated with proteinase K and heat inactivated. Two DNA fragments containing the vox sites and terminal 20 bp homology arms were synthesized (Integrated DNA Technologies, Coralville, Iowa, USA) and inserted into the digested pNG163-R2 using NEBuilder HiFi DNA Assembly according to the manufacturer's protocol (NEB, Ipswich, Mass., USA). Proper insertion of the vox sites was confirmed by Sanger sequencing. Ten μg of the pVoxHV was digested with PacI and transfected into a 6 cm dish of 293 HEK cells using the ProFection Mammalian Transfection System (Promega, Madison, Wis., USA). VoxHV production was performed according to standard protocols (ref). Viral DNA from purified stocks was prepared using the PureLink Viral RNA/DNA Mini Kit (Thermo Fisher, Waltham, Mass., USA).

HdAd mClover3 and HdAd 312 FLEX EGFP Production

The green fluorescent reporter mClover3 (Ref) was cloned into a shuttle plasmid with CMV promoter and SV40 polyA signal and subsequently were transferred to the HdAd plasmid C4HSU (REF) to create pHdAd CMV mClover3. To create pHdAd FLEX EGFP, the FLEX EGFP sequence from Addgene plasmid 59331 (pAAV-CAG-FLEX-EGFP, a gift from Ian Wickersham; http://n2t.net/addegen:59931 RRID:Addgene 5933). The syn FLEX EGFP bgh polyA cassette was then cloned into the HdAd plasmid C4HSU syn mCherry (Lubbert et al., 2017; Lubbert et al., 2019) to create pHdAd syn FLEX EGFP syn mcherry, which contains two separate transgene cassettes for reporter expression.

All HdAd vectors were created using standard protocols. 10 μg of either PmeI digested pHdAd mClover3 or pHdAd FLEX EGFP syn mCherry was transfected into Vika293 cells using the ProFection Mammalian Transfection System (Promega, Madison, Wis., USA) to create HdAd CMV mClover3 or HdAd syn FLEX EGFP syn mCherry viral vectors. Twenty-four hours later, the cells were infected with voxHV and cells were harvested 48 hours post-infection when the cells exhibited full cytopathic effect. HdAd vector was released by three cycles of freeze/thaw and a portion of the cleared cell lysate was used to infect a new dish of Vika293 cells along with voxHV. A total of four serial amplifications was performed with the final passage consisting of thirty 15 cm² dishes. Vector was purified using three sequential cesium chloride gradients and the final prep dialyzed into 250 mM sucrose, 40 mM HEPES pH 7.4, and 1 mM MgCl₂.

### Determination of Viral Titer

Physical titers were determined by spectrophotometry to measure absorbance at 260 nm. Physical titer (vp/ml) was calculated using the following equation: vp/ml=(A260× dilution factor×1.1×10¹²×36)/vector genome size in kb (Ref). Determination of HdAd was determined by FACS of infected HeLa cells. VoxHV infectious titers were determined by AdenoX Rapid Titer Kit (Takara Bio, Mountain View, Calif., USA). Helper virus contamination was measured by digital drop PCR (ddPCR) to quantify viral genomes from infected cells. Briefly, HeLa cells in 6-well dishes were infected with 5 μl purified virus stock for four hours. Cells were washed with PBS and lysed in buffer containing 0.65% NP-40, 150 mM NaCl, and 10 mM Tris HCl pH 8.0. Nuclei 338 were pelleted by centrifugation at 3,000×g for 2 minutes and washed with 1 ml fresh lysis buffer. Nuclei were pelleted again, and the supernatant discarded. Pelleted nuclei were resuspended in 200 μl PBS and DNA was purified using the DNeasy Blood and Tissue Kit (Qiagen, Hilden, DE). DNA was diluted 1:500 and used as template for ddPCR using the following primer/probe sets: HdAd forward 5′-CCCCGCTACCCCAATCC-3′ (SEQ ID NO:7), HdAd reverse 5′-TTAGCTTTTTTGGGTGATTTTTCC-3′ (SEQ ID NO:8), HdAd probe VIC-5′-AGCCTCTCTCATCTCACAGT-3′-MGBNFQ (SEQ ID NO:32), HV forward 5′-TGGGCGTGGTGCCTAAAA-3′ (SEQ ID NO:9), HdAd reverse 5′-GCCTGCCCCTGGCAAT-3′ (SEQ ID NO:11), HV probe FAM-′-TGTCTTTCAGTAGCAAGCT-3′-TAMRA (SEQ ID NO: 33). Template, primers, and probes were mixed with ddPCR™ Supermix for Probes (Bio-Rad Laboratories, Hercules, Calif., USA). Droplets were formed using ddPCR™ Droplet Reader Oil (Bio-Rad Laboratories, Hercules, Calif., USA). Cycling parameters were as follows: initial denaturation at 95° C. for 10 minutes followed by 40 cycles of 94° C. for 30 seconds and 58° C. for 1 minute. A final incubation at 98° C. for 10 minutes was performed prior to droplet count. Droplets were generated and read using the Bio-Rad QX200 Droplet Digital PCR System.

### PCR Excision Assay

Total DNA from crude cell lysates was purified using the PureLink Genomic DNA Mini Kit (Thermo Fisher Scientific, Waltham, Mass., USA). PCR was carried out using EconoTaq Master Mix (Lucigen, Middleton, Wis., USA), 10 ng—DNA template, and the following primers: psi forward 5′ GGAAGTGTGATGTTGCAAGT-3 (SEQ ID NO:12) and psi reverse 5′-CAATGCTGGAGCCCATC-3′ (SEQ ID NO:13). PCR products were run on a 1.5% agarose gel stained with ethidium bromide.

### Western Blot

Cells were lysed in buffer containing 10 mM Tris HCl pH 8.0, 1 mM EDTA, 1% Triton X-100, 0.1% sodium deoxycholate, 0.1% SDS, 140 mM NaCl, and complete protease inhibitor cocktail (Roche, Basel, CH). Lysates were cleared by centrifugation at 20K×g for 30 minutes at 4° C. and protein was quantified using the Pierce BCA assay (Thermo Fisher Scientific, Waltham, Mass., USA). Twenty micrograms of protein was boiled in Laemmli sample buffer (32.9 mM Tris HCl pH 6.8, 13.2% glycerol, 1% SDS, 0.005% bromophenol blue, and 355 mM 2-mercaptoethanol). Proteins were separated using 4-20% tris-glycine PAGE and transferred to PVDF membrane (Bio-Rad Laboratories, Hercules, Calif., USA). Blots were blocked in 5% non-fat dry milk in TBS-T and incubated at 4° C. overnight with 0.5 μg/ml mouse monoclonal anti-HA antibody clone 5B1D10 (Thermo Fisher, Waltham, Mass., USA). A duplicate set of samples on the same blot was incubated with 0.4 μg/ml mouse monoclonal anti-actin antibody clone AC-15 (Sigma-Aldrich, St. Louis, Mo., USA). Blots were then incubated with 0.2 μg/ml goat anti-mouse-HRP, cat. #32230 (Thermo Fisher Scientific, Waltham, Mass., USA) and developed using Clarity Western ECL (Bio-Rad Laboratories, Hercules, Calif., USA).

Infection of 293 and 116 Cells with HdAdsyn FLEX EGFP Syn mCherry

6 cm dishes were seeded with 7.4×10⁵ 293 and 116 cells. Dishes were infected 24 hours later at 70% confluency (2×10⁶cells) with 200 vp/cell of HdAd syn FLEX EGFP syn mCherry. Images were taken 72 hours post infection in the EGFP, mCherry, and bright field channels. An overlay image of the EGFP and mCherry channels was created using Fiji software.

### Stereotactic Surgery

Stereotactic injection of HdAds into the cochlear nucleus (CN) was performed at postnatal day 1 (P1) as previously described in Chen et al. (2013). Briefly, mice were placed in a rubber glove and cooled for 5 minutes in an ice bath and secured on a chilled aluminum block. Depth of anesthesia was checked throughout the surgery by absence of tail pinch response. Surgery took place under aseptic conditions. After the animals were anesthetized a glass needle with tip opening about 20 μm that is filled with viral vector was injected into the cochlear nucleus. For dual HdAd Cre (Lubbert et al., 2017) and HdAd FLEX EGFP injection, vectors were mixed at a 1:1 particle ratio for a final concentration of 1×10⁹ particles/ul. A total of approximately about 1-1.5 and HdAd FLEX EGFP (about 1×10⁷ were injected into C57B⅙J mice. For experiments with Math5 Cre mice, approximately about 1-1.5 HdAd FLEX EGFP (about 1×10⁷ were injected into the CN. In both cases, the viral vector solution was injected at a flow rate of approximately 0.5 μl/min. After injection, the glass needle was left in place for 1 minute to dissipate potential back pressure issues and then slowly removed. Animals were then placed under a warm lamp. Upon full recovery, visual inspection and tail pinch responsiveness, animals were returned to their respective cage with the dam. If animals did not recover from the surgery, they were immediately euthanized. In the case of ranting or if animals were found in distress at later time point (days after surgery) the veterinary staff is consulted and their recommendation for further action was followed.

### Imaging Cell Lines

All microscopic images of cell lines were captured at 10× magnification using an Olympus CKX41 microscope with an EGFP filter set and mCherry filter set equipped with DP21 camera and X-Cite Series 120Q Fluorescence Lamp.

### Imaging Auditory Brainstem

To analyze transduction in the auditory brainstem, brains were dissected out from the injected animals at age postnatal day 9 (P9). Meningeal layers were carefully removed from brain samples, followed by a brief wash in 0.1 M PBS and stored in 4% PFA (in 1×PBS) for 16-24 hours. Brain samples were washed 3 times for 10 minutes with 0.1 M PBS. Coronal auditory brainstem slices of 50 μm thickness containing CN and MNTB from the fixed brain samples were prepared using a Leica VT 1200 vibratome. Free floating slices were incubated with DAPI ml) for about 5 minutes and briefly washed with 0.1 M PBS. Sections then were mounted with Vectashield hardset antifade mounting medium with DAPI (H-1500, Vector laboratories, Burlingame, Calif., USA. To analyze transduction in the auditory brainstem. Confocal images were acquired with a Zeiss LSM 880. Auditory brainstem slices were imaged using z-stack and tilescan mode (bounding grid) with Plan Neofluar 10×/0.3 and 20×/0.8 and online stitched with overlap 10-25%. Emission signal intensity for each channel was adjusted to below the saturation level. Images were further processed in Fiji (Image) 1.53c) and Adobe Photoshop.

### Results

### Development of Vika293 Cell Line.

To develop a Vika/vox-based HdAd production system, a producer cell line was generated that stably expresses high levels of Vika and Ad E1 proteins. HEK293 (293) cells were chosen as the platform since they stably express the Ad E1 proteins, are readily available, and support high levels of Ad replication. The Vika cDNA was codon optimized for human expression and an N-terminal HA epitope tag and nuclear localization signal (NLS) were added to create HA94 NLS-Vika. Then a LVV expressing HA-Vika-NLS was used to generate a pooled population of stably transduced HEK293 cells. To confirm Vika mediated site-specific excision of DNA in the pooled population, a reporter plasmid containing a CMV-vox-dsRed-vox-EGFP cassette (FIG. 1A) was transfected. In the absence of Vika, dsRed is expressed from the CMV promoter, while in the presence of Vika, the dsRed ORF is excised, leading to expression of EGFP. The initial characterization of the pooled population of Vika cells showed that the majority of cells expressed EGFP, indicating that Vika site-specific excision was successful. However, a small but significant fraction of dsRed expressing cells were found, which indicated heterogeneity in Vika excision activity (FIG. 1B). Therefore, to exclude cells with little or no Vika activity, clonal populations of Vika cells were established to identify individual clones with high levels of excision activity. Parental Vika cells were transfected with pCMV-vox-dsRed-vox-EGFP followed by live single-cell fluorescence activated cell sorting (FACS) to select for individual cells that expressed EGFP but not dsRed (FIG. 1B). Clones with high levels of EGFP and undetectable dsRed expression were then amplified. Clones in which the reporter plasmid was found to have integrated into the chromosomal DNA were excluded from further analysis. Based on the screening a clone was identified, Vika 24, which showed almost no dsRed expression compared to the pooled population (FIG. 1B). Western blot analysis for Vika expression demonstrated that this clone had higher levels of Vika expression compared to the pooled population (FIG. 1C). This clone was chosen for subsequent HdAd production and is renamed as Vika293 cells.

Creation and Characterization of voxHV.

To create an HV genome that is unpackageable after Vika excision, the Ad ψ was flanked by vox sites to create a vox HV plasmid, pVoxHV which was transfected into 293 HEK producer cells. VoxHV was amplified and purified according to standard protocols (Palmer & Ng, 2011b). Restriction digest analysis was performed to confirm the integrity of the voxHV genome (FIG. 2A). Both plasmid DNA and viral DNA from purified viral particles were digested with MfeI to distinguish the circular plasmid DNA from replicated linear viral DNA. Both the pVoxHV and VoxHV DNA show the expected banding patterns (FIG. 2A). VoxHV grew to a physical titer of 3.5×10¹² viral particles (vp)/ml with an infectious titer of 2.1×10¹¹ infectious units (IU)/ml. These values are similar to titers obtained using the loxP HV (NG163-R2) (Palmer & Ng, 2003). Taken together, these data demonstrate that the vox sites have no effect on viral DNA replication, assembly, or infectivity of the HV.

### VikAD Production of HdAd.

An effective HdAd production system must support production of HdAd to high titers that contain low levels of contaminating HV. To test the efficiency of the VikAD production system, an HdAd CMV mClover3 vector was amplified. Crude lysates were examined at each passage of HdAd production for both excision of VoxHV and HdAd amplification. To evaluate excision of the ψ from VoxHV in Vika293 cells, standard non-quantitative PCR using 30 cycles of amplification and primers that flank the from VoxHV was performed (FIG. 2B). As a positive control for excision, 116 (Cre-expressing) cells were infected with the loxP HV (Palmer & Ng, 2003). As a negative control for excision, 293 cells were infected with the voxHV. Based on our PCR excision assay we were unable to detect VoxHV genome with the intact at any passage of HdAd amplification.

To assess the efficiency of HdAd amplification, a portion of the crude lysate from an equal number of Vika293 cells at each passage was used to transduce 293 cells. Images were captured 24 hours post-transduction to monitor expression of the mClover3 reporter from the HdAd vector (FIG. 2C). A dramatic increase in mClover3 signal was observed at each HdAd amplification passage as evidenced by the relative number of cells expressing mClover3 and the decrease in the exposure times necessary to visualize transduced cells. Analysis of HdAd titer revealed a physical titer of 2.0×10¹² viral particles (vp)/ml and an infectious titer of 1.0×10¹¹ infectious units (IU)/ml. These values are similar to the titers of an identical HdAd CMV mClover3 vector that was produced using the loxP HV and 116 cells using an identical vector amplification protocol, which grew to 2.3×10¹² viral (vp)/ml with an infectious titer of 5.0×10¹⁰ (IU)/ml).

Production of HdAd Containing loxP Sites.

Genetic intersectional strategies relying on Cre or Flp have become tools in understanding cellular and molecular mechanisms of cellular network activity (Weissman & Pan, 2015; Atasoy et al., 2008). Since VikAD produced high titer HdAd, it was determined if an HdAD virus could be prepared that was dependent on Cre-mediated excision for transgene expression. To do so, FLEX EGFP (Addgene #59331, gift of Dr. Ian Wickersham) under the control of the hsyn promoter was cloned into an HdAd genomic plasmid that also contained the hsyn mCherry transgene expression cassette (Lubbert et al., 2017) (FIG. 3A). HdAd FLEX EGFP virus was produced and purified to high titers using standard methods.

To determine the excision efficiency of the VikaAD system during amplification, additional characterization of HdAd FLEX EGFP was conducted to determine HV contamination prior to density purification. Although excision of the in the VoxHV was comparable to the commonly used loxP HV when measured by non-quantitative PCR, it is well known from the Cre and Flp production systems that excision efficiency is not 100% and that there are differences in excision efficiencies between the Cre and Flp producer cell lines (Palmer & Ng, 2003; Umana et al., 2001; Ng et al., 2001; Parks et al., 1996). Therefore, to determine Vika excision efficiency on the VoxHV, ddPCR was carried out on crude lysates of HdAd FLEX EGFP prior to final purification. Crude lysate from the final passage contained approximately 0.4% HV contamination, which is similar to previous reports of HV contamination with Cre and Flp production systems prior to density gradient purification (Palmer & Ng, 2003; Umana et al., 2001; Ng et al., 2001). Based on these results, our VikAD system mediates efficient production of HdAd with HV contamination levels comparable to the Cre and Flp systems.

VikaAd Enables the Production of HdAd Vectors with Cre Excision-Dependent Transgene Expression.

To determine if Cre-mediated excision led to EGFP expression from HdAd FLEX EGFP in an in vitro context, we transduced 116 cells (Palmer & Ng, 2003) (Cre+) or 293 cells (Cre−). Although the hSyn promoter is a neurospecific promoter (Kugler et al., 2001), it has low levels of activity in 293 cells and their derivatives as 293 are related to neuronal cells (Shaw et al., 2002). Analysis of the 116 and 293 cells 72 hours after transduction revealed that 293 cells only expressed mCherry (FIG. 3B). However, 116 cells that were mCherry positive also expressed EGFP, demonstrating efficient Cre-mediated inversion of the EGFP sequence. To test functionality in vivo with a mouse Cre driver line in a native neuronal context, we injected HdAd FLEX EGFP into the P1 mouse cochlear nucleus (CN) (Chen et al., 2013) of the Math5 mouse Cre driver line or C57BL/6J (wild-type, wt) mouse line (FIG. 4A). Math5 (Atoh7) is a transcription factor that leads to selective labeling of the globular bushy cells (GBCs) in the CN (Saul et al., 2008; Xu et al., 2016). Analysis of the P9 auditory brainstem revealed EGFP expression in the CN, the GBC axons, and the calyces of Held, large glutamatergic presynaptic terminals which arise from the GBCs axon in the Math5 Cre mouse line but not wild-type. (FIG. 4B, C, D). Finally, to test whether co-delivery of HdAd FLEX EGFP with an HdAd Cre vector leads to efficient EGFP expression, an HdAd Cre, which contains an hsyn mCherry reporter (Lubbert et al., 2017), was co-injected with HdAd FLEX EGFP into the P1 mouse CN (FIG. 5A). Analysis of the P9 auditory brainstem revealed EGFP expression that co-localized with EGFP expression in the CN and axons that projected from the CN (FIG. 5B). In particular, the calyces of Held were co-labeled with EGFP and mCherry in the contra-lateral medial nucleus trapezoid body (MNTB) from the injection site (FIG. 5C, D). Based on these results from cell lines, mouse Cre driver line, and dual viral injections, VikAD leads to effective production of an HdAd vector for cell-type specific labeling using Cre-dependent expression for in vitro and in vivo applications.

### DISCUSSION

By generating a cell line that expresses Vika and an Ad HV that contains the flanked by vox sites, an HdAd production platform, VikAD, was created that enables the use of HdAd with genetic intersectional strategies using Cre and Flp or other site specific recombinases in vitro and in vivo. Therefore, VikAD fills an important need for intersectional genetic strategies for cell-type specific expression of large genes and transgene expression cassettes not possible with rAAV or LVV.

### VikAD and HdAd Production

The PCR excision assay revealed similar excision efficacy between 116 and Vika293 cells and an analysis of HV contamination of crude lysate during VikAd production showed that Vika293 produced HdAd with a similar level of HV contamination as the 116 and Flp producer cell lines (Palmer & Ng, 2003; Umana et al., 2001; Ng et al., 2001). This demonstrates that VikAD is as efficient as these producer systems in excising the Y. However, VikAD may be improved with regard to HV contamination prior to purification. A previous study showed that the increased levels of Cre recombinase in 116 cells compared to 293NS4 cells lead to significant increases in excision efficiency with a significant reduction in HV contamination prior to density gradient purification (Palmer & Ng, 2003). Therefore, the excision efficacy in Vika293 cells may be improved by creating additional clonal cell lines with higher levels of Vika expression or by delivery of additional Vika coding sequence to the current cell line. Although Vika has significantly lower toxicity compared to Cre22, Vika expression may be at maximal levels in Vika293 cells, since Vika is codon optimized for expression in human cells. Another possibility to determine if Vika excision activity is increased would be to use molecular evolution techniques (Yang et al., 2019). This strategy has been successfully used to create Flp recombinases with high thermostability and more efficient Flp excision (Buchholz et al., 1998). Finally, HV contamination might be further reduced by altering the spacing of the between the vox sites or the distance between the ITR and vox Y vox site (Hearing et al., 1987).

VikAD for Use with Genetic Intersectional Strategies

VikAd enabled the production an HdAd FLEX EGFP vector that enables cell-type specific expression in the context of Cre in vitro and in vivo. Vika is extremely specific and does not cross-recombine on Frt, Cre and Cre-derivative, or noxP and pox sites (Karimova et al., 2013; Karimova et al., 2016). Therefore, VikAD enables the production of HdAd vectors that utilize Cre, Flp, or Cre-derivative sites for transgene expression (Atasoy et al., 2008; Fenno et al., 2017; Fenno et al., 2020) to permit cell-type specific and sparse labeling of cells in animals. In addition, since HdAd can package up to 36 kb of foreign DNA, VikAD can be used to create HdAd vectors that utilize these recombinase-dependent expression cassettes to express cDNAs, sensors, or other constructs that are too large to be packaged into AAV and LVV. Co-delivery of HdAd Cre with the HdAd FLEX EGFP vector leads to Cre-dependent reporter expression, showing that VikAD enables the ability to exploit the advantages of HdAd in combination with cell-type specific rAAV and LVV expression vectors to probe neuronal circuit function across multiple animal species. While Ad Type 5 vectors have broad tropism, there are cell-types that are refractory to Ad Type 5 but not rAAV or LVV. The viral vectors in a dual-injection approach should have overlapping tropism. Taken together, VikAD is an important addition to the viral vector field that increases the number of tools for elucidating the cellular and molecular mechanisms controlling cellular activity in vivo and regulation of organismal behavior.

### REFERENCES

Anguela & High, Annu. Rev. Med., 70:273 (2019).
Atasoy et al., J. Neuroscience 28:7025 (2008).
Brunetti-Pierri & Ng, Virus Genes, 53:684 (2017).
Buchholz et al., Nature. Biot., 16:657 (1998).
Chen et al., Curr. Protoc. Mouse Biol., 8:e58 (2018).
Chen et al., J. Neuroscience, 33:8336 (2013).
Fenno et al., Cur. Prot. Neuroscience, 80: (2017).
Fenno et al., Neuron, 107:836 (2020).
Gilbert et al., Hum. Mol. Genet., 12:1287 (2003).
Hearing et al., J. Virol., 61:2555 (1987).
Karimova et al., Nucleic Acids Res., 41:e37 (2013).
Karimova et al., Sci. Rep., 6:30130 (2016).
Kiang et al., Mol. Ther., 13:127 (2006).
Kim et al., Proc. Natl. Acad. Sci. USA, 98:13282 (2001).
Kovesdi & Hedley, Viruses. 2:1681 (2010).
Kugler et al., Mol. Cell. Neurosci., 17:78 (2001).
Li & Samulski, Genetics, 21:255 (2020).
Lubbert et al., eLife, 6:28412 (2017).
Lubbert et al., Neuron, 101:260 (2019).
Mian et al., Mol. Ther., 10:492 (2004).
Miura et al., Nat. Protoc., 13:195 (2018).
Nectow & Nestler, Nat. Rev. Neurosci., 21:669 (2020).
Ng et al., Mol. Ther., 3:809 (2001).
Oka et al., Gene Ther., 14:191 (2007).
Palmer & Ng, Cold Spring Harbor Protocols, pg. 867 (2011a).
Palmer & Ng, Cold Spring Harbor Protocols, pg. 867 (2011b).
Palmer & Ng, Mol. Ther., 8:846 (2003).
Parks et al., Proc. Natl. Acad. Sci. USA, 93:13565 (1996).
Pinkert, Transgenic Animal Technology, 2^{nd} Ed. (Elsevier Press, 2002).
Rastall et al., Gene Ther., 23:743 (2016).
Saul et al., Mol. Cell. Neurosci., 37:153 (2008).
Schmitt et al., Human Gene Ther. Meth., 25:181 (2014).
Shaw et al., FASEB J. 16:869 (2002).
Toietta et al., Proc. Natl. Acad. Sci. USA, 102:3930 (2005).
Umana et al., Nature Biot., 19:582 (2001).
Weissman & Pan, Genetics, 199:293 (2015).
Xu et al., J. Neurosci., 36:4408 (2016).
Yang et al., Nature Meth., 16:687 (2019).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. An isolated mammalian cell that stably expresses a recombinant DNA comprising an open reading frame encoding a recombinase having at least 80% amino acid sequence identity to SEQ ID NO:1 and optionally expresses at least one adenovirus protein.

2. The isolated cell of claim 1 wherein the recombinase has at least 90% amino acid sequence identity to any one of SEQ ID Nos. 20-25.

3. The isolated cell of claim 1 or 2 which is a primate cell.

4. The isolated cell of claim 1, 2 or 3 which is a human cell.

5. The isolated cell of any one of claims 1 to 4 wherein the open reading frame further encodes a nuclear localization signal.

6. The isolated cell of any one of claims 1 to 5 which stably expresses at least one adenovirus protein.

7. The isolated cell of any one of claims 1 to 6 wherein the adenovirus protein includes adenovirus H A, E1B or both.

8. A recombinant helper adenovirus vector comprising an adenovirus genome comprising two inverted terminal repeats (ITRs) and a packaging signal flanked by a site-specific recombinase recognition site having at least 80% nucleic acid sequence identity to SEQ ID NO:4.

9. The vector of claim 8 wherein the recombinase recognition site has at least 90% nucleic acid sequence identity to SEQ ID NO:4.

10. The vector of claim 8 wherein the recombinase recognition site has at least 95% nucleic acid sequence identity to SEQ ID NO:4.

11. The vector of claim 8, 9 or 10 wherein the recombinase recognition site is cleaved by a recombinase having at least 80% amino acid sequence identity to SEQ ID NO:1.

12. The vector of any one of claims 8 to 11 wherein the vector is a plasmid.

13. A method to produce helper dependent adenovirus, comprising:
contacting an isolated mammalian cell that stably expresses a recombinase having at least 80% amino acid sequence identity to SEQ ID NO:1, a recombinant helper adenovirus vector comprising two ITRs and a packaging signal flanked by a pair of site-specific recombinase recognition sites having at least 80% nucleic acid sequence identity to SEQ ID NO:4, and a helper dependent adenovirus vector comprising a genome having two ITRs, a packaging signal, an open reading frame for a gene product of interest and a transcriptional control sequence, wherein if the open reading frame is in reverse orientation relative to the transcriptional control sequence, the open reading frame is flanked by a pair of site-specific recombinase recognition sites other than sites having at least 80% nucleic acid sequence identity to SEQ ID NO:4 or other than sites recognized by a polypeptide having at least 80°/o amino acid sequence identity to SEQ ID NO:1 or if the transcriptional control sequence is in reverse orientation relative to the open reading frame, the transcriptional control sequence is flanked by a pair of site-specific recombinase recognition sites other than sites having at least 80% nucleic acid sequence identity to SEQ ID NO:4 or other than sites recognized by a polypeptide having at least 80% amino acid sequence identity to SEQ ID NO:1; and
collecting helper dependent adenovirus.

14. The method of claim 13 wherein the cell is contacted with the recombinant helper adenovirus vector and then with the helper dependent adenovirus vector.

15. The method of claim 13 or 14 wherein the site-specific recombinase recognition sites other than sites having at least 80% nucleic acid sequence identity to SEQ ID NO:4 are loxP, rox, vloxP, pox or nox sites.

16. The method of any one of claims 13, 14 or 15 wherein the transcriptional control sequence is a cell-specific transcriptional control sequence.

17. The method of any one of claims 13 to 16 wherein the open reading frame encodes a protein.

18. The method of any one of claims 13 to 16 wherein the open reading frame encodes a therapeutic gene product.

19. The method of any one of claims 13 to 16 wherein the open reading frame comprises a marker.

20. A method to produce helper dependent adenovirus, comprising:
contacting an isolated mammalian cell that stably expresses a recombinase having at least 80% amino acid sequence identity to SEQ ID NO:1, a recombinant helper adenovirus vector comprising two ITRs and a packaging signal flanked by a site-specific recombinase recognition site having at least 80% nucleic acid sequence identity to SEQ ID NO:4, and a helper dependent adenovirus vector comprising a genome having two ITRs, a packaging signal, an open reading frame for a gene product of interest and a transcriptional control sequence; and
collecting helper dependent adenovirus.

21. A kit comprising two or more of:
a) an isolated mammalian cell that stably expresses a recombinase having at least 80% amino acid sequence identity to SEQ ID NO:1;
b) a recombinant helper adenovirus vector comprising at least a portion of an adenovirus genome comprising two adenovirus ITRs and an adenovirus packaging signal flanked by a pair of site-specific recombinase recognition sites having at least 80% nucleic acid sequence identity to SEQ ID NO:4; and
c) a helper dependent adenovirus vector comprising two ITRs, a packaging signal helper dependent adenovirus vector comprising a genome having two adenovirus ITRs, an adenovirus packaging signal flanked by a pair of site-specific recombinase recognition sites other than sites having at least 80% nucleic acid sequence identity to SEQ ID NO:4 or sites other than ones recognized by a polypeptide having at least 80% amino acid sequence identity to SEQ ID NO:1, and one or more restriction enzyme sites for insertion of a desired DNA fragment.
